(19) United States
(12) Patent Application Publication
(10) Pub. No.: US 2021/0386604 A1
(43) Pub. Date: Dec. 16, 2021

(54) COLLAPSIBLE ISOLATION SYSTEM AND METHOD OF USE

(71) Applicant: Prep Tech, L.L.C., Westlake, LA (US)

(72) Inventors: Mark R. Moore, Westlake, LA (US); Peter Prados, Lafayette, LA (US)

(73) Assignee: Prep Tech, L.L.C., Westlake, LA (US)

(21) Appl. No.: 17/303,260

(22) Filed: May 25, 2021

Related U.S. Application Data
(63) Continuation-in-part of application No. 17/302,232, filed on Apr. 27, 2021.
Application No. 17/302,232 is a continuation-in-part of application No. 17/301,698, filed on Apr. 12, 2021.
Application No. 17/301,698 is a continuation-in-part of application No. 17/301,217, filed on Mar. 29, 2021.
(60) Provisional application No. 62/704,192, filed on Apr. 27, 2020.
(60) Provisional application No. 62/704,385, filed on May 7, 2020.
(60) Provisional application No. 62/705,297, filed on Jun. 20, 2020.
(60) Provisional application No. 62/706,393, filed on Aug. 13, 2020.
(60) Provisional application No. 63/008,331, filed on Apr. 10, 2020.
(60) Provisional application No. 62/704,093, filed on Apr. 21, 2020.
(60) Provisional application No. 63/001,379, filed on Mar. 29, 2020.
(60) Provisional application No. 62/704,092, filed on Apr. 21, 2020.

(51) Int. Cl. A61G 10/00; A61G 10/02

(52) U.S. Cl. CPC A61G 10/005; A61G 10/023

(57) ABSTRACT

A medial isolette is provided. A flexible enclosure and a collapsible wire-frame structure are provided which supply an economical and easily assembled isolation chamber. A flexible drape is provided to further isolate the patient and effectively control a negative pressure environment within the isolette. Access ports with integrated or removable gloves are provided to access the patient when the isolette is in use. A component access panel is provided for ducted connection of patient gas circuits and leads and source of negative pressure.

### CROSS-REFERENCE TO RELATED APPLICATIONS

This application is a continuation-in-part of U.S. application Ser. No. 17/302,232 filed on Apr. 27, 2021, which claims priority benefit from U.S. Provisional Application No. 62/704,192, filed on Apr. 27, 2020; U.S. Provisional Application No. 62/704,385, filed on May 7, 2020; U.S. Provisional Application No. 62/705,297 filed on Jun. 20, 2020 and U.S. Provisional Application No. 62/706,393 filed on Aug. 13, 2020, which is a continuation-in-part of U.S. application Ser. No. 17/301,698 filed on Apr. 12, 2021, which claims priority benefit from U.S. Provisional Application No. 63/008,331, filed on Apr. 10, 2020; and U.S. Provisional Application No. 62/704,093, filed on Apr. 21, 2020, which is a continuation-in-part of U.S. application No. 17/301,217, filed on Mar. 29, 2021, which claims priority benefit from U.S. Provisional Application No. 63/001,379, filed on Mar. 29, 2020, and U.S. Provisional Application No. 62/704,092, filed on Apr. 21, 2020. The patent applications identified above are incorporated here by reference in their entirety to provide continuity of disclosure.

### FIELD OF THE INVENTION

The present invention relates to medical isolettes.

### BACKGROUND OF THE INVENTION

Patient isolation represents one of several measures that can be taken to control the spread of infection and highly contagious diseases. The goal of patient isolation is to contain airborne contagions produced by the patient, or alternatively, to limit airborne contagions to which the patient is exposed.

The U.S. Centers for Disease Control and Prevention (CDC) issues, and periodically revises, recommendations for various levels of patient isolation for disease control. Isolation is typically recommended when a patient is known to have a highly contagious viral or bacterial illness.

Special isolation protocols are used in the management of patients in isolation. Typical isolation protocols include mandating personal protective equipment such as gowns, masks, and gloves and applying physical controls, such as negative pressure rooms, laminar air flow and structural barriers. Negative pressure is used to reduce risk of exposure to airborne contagions produced by the patient.

Patients can produce airborne contagions in any number of circumstances. For example, airborne contagions are typically produced by a patient during intubation. Intubation is required when the patient cannot maintain their airway or cannot breathe without assistance. Intubation requires inserting an endotracheal tube, through the mouth and then into the airway. The tube is then connected to a ventilator. Intubation typically takes place after a patient is anaesthetized. Even so, patient reflex reactions usually cause coughing during the intubation procedure which can result in a significant amount of airborne contagion being released.

The requirement for patient isolation and intubation occurs in many circumstances. For example, patient isolation and intubation can take place in an emergency situation in the field. Emergency isolation in the field is difficult because physical control protocols such as negative pressure rooms and structural barriers are difficult to erect quickly and at low cost. Further exacerbating the problem, intubation is typically prescribed only once a dangerous condition is recognized. Oftentimes, such diagnoses are made in emergencies with extremely limited time to react.

Emergency situations, such as epidemics, also create special problems. For example, epidemics, such as swine flu, MERS-COV, Ebola and COVID-19 are considered highly contagious. Such highly contagious diseases certainly require effective negative pressure isolation and many times also require patient intubation, simultaneously. Effective negative pressure containment is critical to control rapid transmission of these diseases. In these situations, it is extremely difficult to protect caregivers during the intubation process, given the current state of the art in isolation barriers.

The prior art has attempted many different methods and structures to solve the problems of simple, inexpensive and portable isolation barriers but all have fallen short.

For example, U.S. Pat. No. 5,832,919 issued to Kano discloses a portable enclosure system which provides a suitable breathing atmosphere within a chamber, by use of positive pressure provided by fans and exhaust ports. However, the chamber does not allow access to the patient to provide medical treatment and fails to provide a negative pressure environment.

U.S. Pat. No. 7,757,689 issued to Chang discloses an inflatable isolation “cabin” for isolation of the entire patient, prior to transport. However, the cabin must be removed to provide medical treatment such as surgery or intubation.

Another example is disclosed in U.S. Pat. No. 4,949,714, to Orr. Orr discloses a medical hood for fitting over the head of a patient. A gas port leads to the interior of the hood for supplying respiratory gas. An outlet connects the interior of the hood and the ambient air for discharge of gas. However, the hood of Orr is rigid and therefore does not provide for a collapsible frame for ease of use and transport.

Another example is U.S. Pat. No. 5,950,625 to Bongiovanni, et al. Bongiovanni discloses an isolation bag for isolating a casualty from a contaminated environment. The bag is fabricated from a transparent biochemically resistive material and includes a self-contained transportable life support system. However, the transportable system, including the bag, is difficult to replicate cheaply or transport easily and is time consuming to assemble and use.

A need exists to provide a device and method to isolate a patient while allowing the patient to be an intubated or receive other medical treatment. Further, a need exists for an isolation chamber that may be quickly deployed to protect health care providers from contagions while completing the intubation procedure. Still further, a need exists for an isolette that is inexpensive and which can be easily and quickly mass produced to aid in mass isolation of patients with highly contagious epidemic diseases.

A further need exists for an isolette that provides a negative pressure environment to protect health care workers when accessing a patient.

### SUMMARY OF THE INVENTION

A preferred embodiment includes a collapsible rigid frame that supports a flexible enclosure sufficient to enclose the head and upper torso of a patient. The frame includes two side supports and a front support that are rotatably connected to a base frame. In one embodiment, the flexible enclosure includes a bottom panel, two side panels, a front panel, a top panel and an integrated drape. Each side panel may include one or more access ports. The front panel accommodates two side-by-side access ports.

In a preferred embodiment, the access ports can further include integrated gloves, resealable flaps, sealing rings, or iris diaphragms. In another preferred embodiment, the access ports can include removable gloves fixed in the access ports by separable fittings or collars.

A component access panel is provided in either or both of the side panels which serves as a pass through for a turbine negative pressure unit, ventilation tubes, and patient support leads. In one preferred embodiment, the component access panel includes removable plugs of various sizes that are fixed in the access panel by microperforations. When the plugs are removed, passages of various sizes are created that accommodate tubes and electrical leads.

In another preferred embodiment, the component access panel is provided with various connectors that accommodate a ventilation circuit, a Luer Lock connector or other connectors for a CO₂ monitor.

Another preferred embodiment of a collapsible rigid frame includes two side supports and a front support, positioned by a set of removable cross braces. The side supports are each rotatably connected to the front support. The cross braces are movably attached to the two side supports.

The drape subassembly is constructed from cost effective polyethylene sheeting. The drape includes an optional flexible seal and a strap for securing the drape around the patient.

To prepare the isolette for use, the isolette is assembled by unfolding the rigid frame from a collapsed arrangement to a deployed arrangement. The frame is inserted into a flexible enclosure. The drape of the flexible enclosure is positioned on top of the isolette. A patient is positioned inside the isolation chamber formed by the enclosure isolette and the drape is extended along the patient's torso. The flexible seal is positioned transversely across the patient and secured by the strap. A negative pressure is supplied to the isolette by a turbine connected to the isolette through a pressure port. Access to the patient while in the isolette, is provided by the gloves sealed into conveniently positioned access ports.

### BRIEF DESCRIPTION OF THE DRAWINGS

In the detailed description of the preferred embodiments presented below, reference is made to the accompanying drawings. Unless otherwise specified, all tolerances are ±20%.

FIG. 1 is an isometric view of a preferred embodiment of an isolette.

FIG. 2 is an isometric view of a preferred embodiment of an enclosure subassembly.

FIG. 3A is an exploded isometric view of the preferred embodiment of a frame subassembly.

FIG. 3B is an isometric view of a preferred clip.

FIG. 3C is a detail isometric view of a preferred embodiment of a rotational connection.

FIG. 4 is a plan view of a preferred embodiment of a drape subassembly.

FIG. 5A is a plan view of the preferred embodiment of a frame subassembly in a collapsed position.

FIG. 5B is an isometric view of the preferred embodiment of a frame subassembly.

FIG. 5C is an isometric view of the preferred embodiment of a frame subassembly in a deployed position.

FIG. 5D is an exploded isometric view of the preferred embodiment of an isolette.

FIG. 5E is a flowchart of a preferred method of assembly of an isolette.

FIG. 6 is a detail view of an alternate preferred embodiment of a side panel.

FIG. 7 is a detail view of an alternate preferred embodiment of a side panel.

FIG. 8A is an exploded view of a preferred component access panel.

FIG. 8B is an exploded view of an alternate embodiment of an access panel.

FIG. 9 is an isometric view of an alternate embodiment of a component access panel.

FIG. 10A is an exploded isometric view of a preferred glove connection.

FIG. 10B is an isometric view of a preferred glove connection.

FIG. 10C is an isometric view of a preferred glove connection.

FIG. 10D is an exploded isometric view of a preferred glove connection.

FIG. 10E is an exploded isometric view of a preferred barrier cap.

FIG. 11 is a side view of a preferred embodiment of an isolette in use.

FIG. 12A is an exploded isometric view of a preferred embodiment of a frame subassembly.

FIG. 12B is an isometric view of a preferred connector.

FIG. 12C is a detail isometric view of a preferred rotational connection.

FIG. 12D is a detail isometric view of a preferred slidable connection.

FIG. 13A is a plan view of a preferred embodiment of a frame subassembly in a collapsed position.

FIG. 13B is an isometric view of a preferred embodiment of a frame subassembly.

FIG. 13C is an exploded isometric view of a preferred embodiment of a frame subassembly in a deployed position.

FIG. 13D is a flowchart of a preferred method of assembly of an isolette.

FIG. 14 is a flowchart of a preferred method of use of an isolette.

### DETAILED DESCRIPTION OF THE INVENTION

In the description that follows, like parts are marked throughout the specification and figures with the same numerals, respectively. The figures are not necessarily drawn to scale and may be shown in exaggerated or generalized form in the interest of clarity and conciseness.

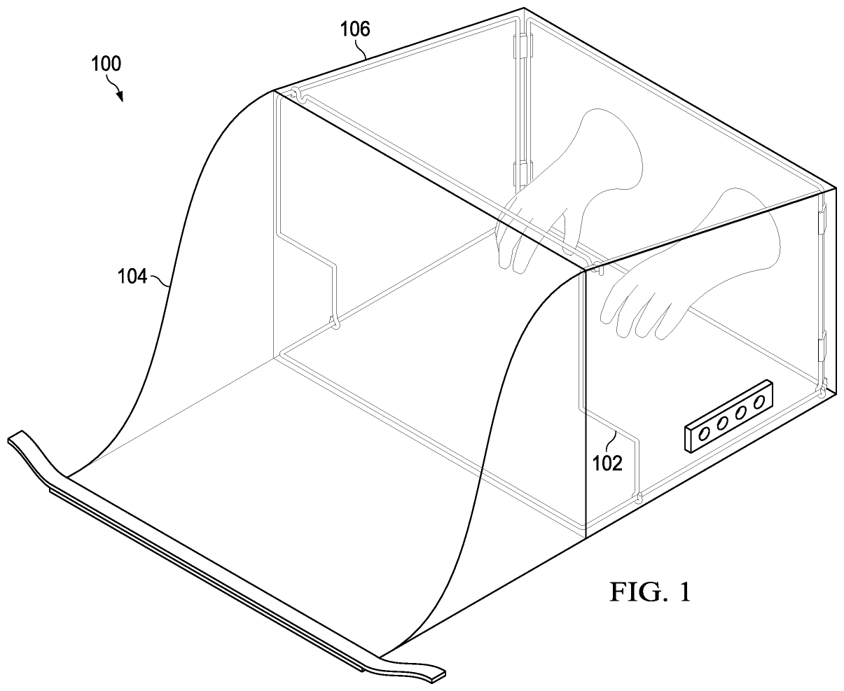

Referring to FIG. 1, isolette 100 will be further described. A preferred embodiment of isolette 100 includes frame subassembly 102, enclosure subassembly 106, and drape subassembly 104.

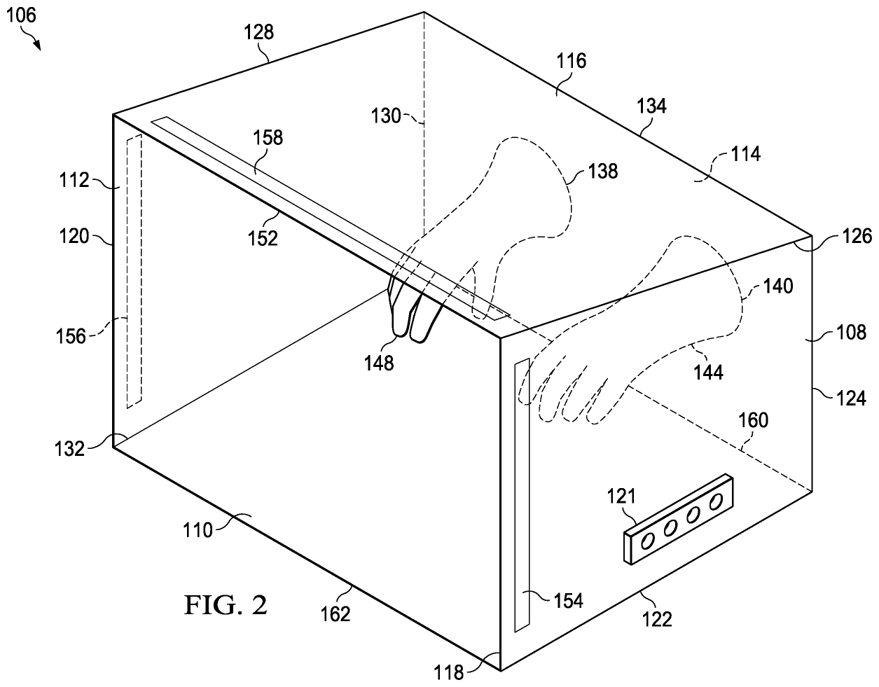

Referring to FIG. 2, enclosure subassembly 106 will be further described. Enclosure subassembly 106 includes side panel 108, side panel 112, front panel 114, bottom panel 110, and top panel 116. Enclosure subassembly 106 is preferably comprised of transparent polyethylene sheeting with a thickness of between about 8 and 30 mil. Other generally transparent non-porous flexible or semi-flexible materials will suffice.

Side panel 108 is bounded by rear edge 118, angled edge 126, front edge 124, and bottom edge 122. Side panel 108 includes component access panel 121 which will be further described. Adhesive strip 154 is positioned along rear edge 118. In an alternate embodiment, side panel 108 may also include one or two access ports (not shown) and gloves (not shown), as will be further described.

Side panel 112 is bounded by rear edge 120, angled edge 128, bottom edge 132, and front edge 130. Adhesive strip 156 is attached along rear edge 120. In an alternate embodiment, side panel 112 may also include one or two access ports (not shown) and gloves (not shown), as will be further described.

Front panel 114 is bounded by top edge 134, bottom edge 160, front edge 124, and front edge 130. Front panel 114 includes access ports 138 and 140. Glove 148 is adhered to the outside face of front panel 114, within access port 138. Glove 144 is adhered to the outside face of front panel 114 at access port 140.

Gloves 144 and 148 are bi-directional, that is, they may be used with either hand and are positioned in the access ports with the thumb directed upward with respect to the isolette.

Top panel 116 is bounded by rear edge 152, angled edge 126, top edge 134, and angled edge 128 and adhesive strip 158 is attached along rear edge 152, as will be further described.

Bottom panel 110 is bounded by rear edge 162, bottom edge 122, bottom edge 160 and bottom edge 132.

Side panel 108 is attached to top panel 116 along angled edge 126, front panel 114 along front edge 124, and bottom panel 110 along bottom edge 122. Side panel 112 is attached to top panel 116 along angled edge 128, front panel 114 along front edge 130, and bottom panel 110 along bottom edge 132. Front panel 114 is further attached to bottom panel 110 along bottom edge 160 and top panel 116 along top edge 134. Preferably, side panels 108 and 112, front panel 114, and bottom panel 110 are integrally formed. Optionally, the panels may be inductively welded together.

Bottom edges 122 and 132 are preferably about 16 inches in length. Rear edge 162, rear edge 152, top edge 134 and bottom edge 160 are preferably about 24¾ inches in length. Rear edges 118 and 120 are preferably about 18¼ inches in length. Angled edges 126 and 128 are preferably about 16¼ inches in length. Front edges 124 and 130 are preferably about 15½ inches in length. The difference in the lengths between rear edges 120 and 118 and front edges 124 and 130 imparts a downward sloping angle to top panel 116 of about 7° when the isolette is assembled which aids in viewing the patient through top panel 116. Other dimensions will suffice.

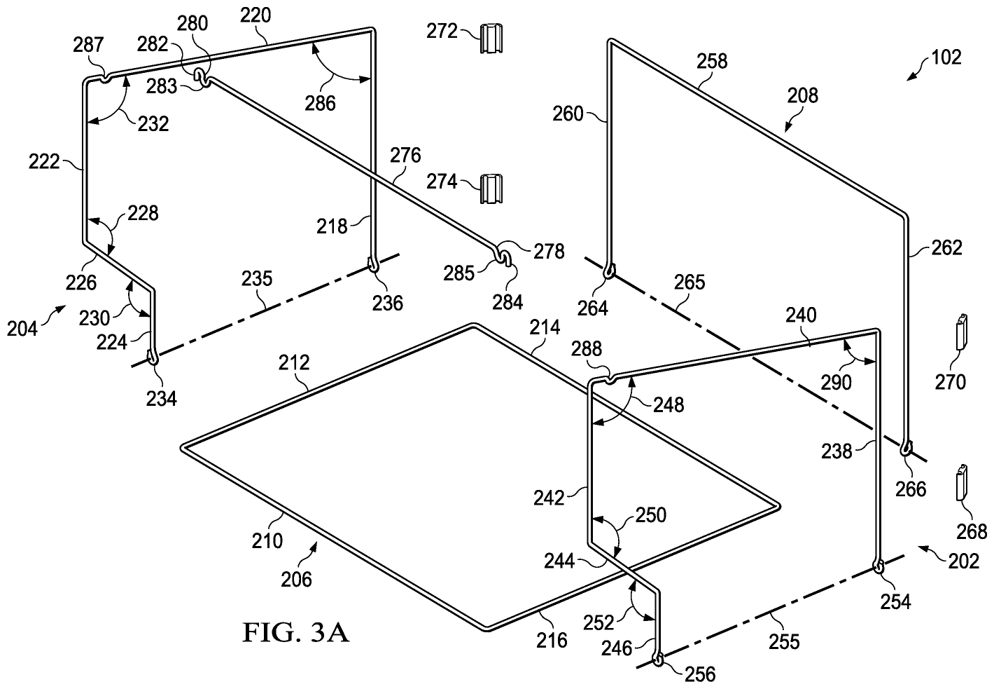

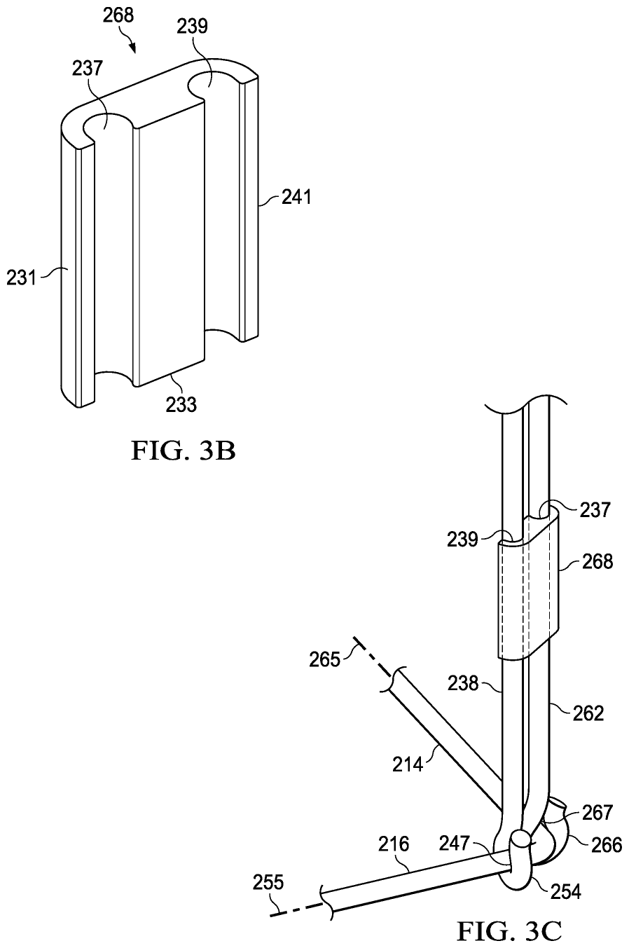

Referring to FIGS. 3A and 3B, frame subassembly 102 will be further described. Frame subassembly 102 includes base support frame 206, support frame 202, support frame 204, front support frame 208, and cross brace 276. Preferably the components of frame subassembly 102 are constructed from approximately 3/16 inch diameter stainless steel or aluminum drawn wire or lightweight hollow metallic tubing. Alternatively, rigid fiberglass or graphite composite rod will also suffice. Also, alternatively a mild steel drawn wire having a painted or powder coated finish will also suffice.

Base support frame 206 includes side rails 212 and 216, rear rail 210 and front rail 214. Preferably the side rails are about 15 inches in length and rear rail 210 and front rail 214 are preferably about 24.4 inches in length.

Support frame 202 includes front stanchion 238, top rail 240, rear stanchion 242, angled segment 244, and vertical segment 246. Vertical segment 246 includes hook 256. Front stanchion 238 includes hook 254. The hooks are coaxial along axis 255 and are dimensioned to rotatably engage side rail 216, as will be further described. Axis 255 is generally perpendicular to vertical segment 246 and front stanchion 238. Top rail 240 includes indention 288 adjacent rear stanchion 242. Front stanchion 238 is connected to top rail 240 at angle 290 which is preferably about 97°. Top rail 240 is connected to rear stanchion 242 at angle 248 which is preferably about 83°. Rear stanchion 242 is connected to angled segment 244 at angle 250 which is preferably about 135°. Angled segment 244 is connected to vertical segment 246 at angle 252 which is preferably about 135°. During assembly, hooks 256 and 254 are positioned around side rail 216 to connect support frame 202 to base support frame 206, but allow support frame 202 to rotate with respect to side rail 216. In a preferred embodiment, all the components of support frame 202 are integrally formed.

Support frame 204 includes front stanchion 218, top rail 220, rear stanchion 222, angled segment 226, and vertical segment 224. Vertical segment 224 includes hook 234. Front stanchion 218 includes hook 236. The hooks are coaxial along axis 235 and are dimensioned to rotatably engage side rail 212, as will be further described. Axis 235 is generally perpendicular to vertical segment 224 and front stanchion 218. Top rail 220 includes indention 287 adjacent rear stanchion 222. Front stanchion 218 is connected to top rail 220 at angle 286 which is preferably about 97°. Top rail 220 is connected to rear stanchion 222 at angle 232 which is preferably about 83°. Rear stanchion 222 is connected to angled segment 226 at angle 228 which is preferably about 135°. Angled segment 226 is connected to vertical segment 224 at angle 230 which is preferably about 135°. During assembly, hooks 234 and 236 are positioned around side rail 212 to connect support frame 204 to base support frame 206, but allow support frame 204 to rotate with respect to side rail 212. In a preferred embodiment, the components of support frame 204 are integrally formed.

In a preferred embodiment, front stanchions 218 and 238 are about 15 inches in length, top rails 220 and 240 are about 15 inches in length, rear stanchions 222 and 242 are about 9 inches in length, angled segments 226 and 244 are about 6 inches in length, and vertical segments 224 and 246 are about 4 inches in length.

Front support frame 208 includes side rail 260, top rail 258, and side rail 262. Side rail 260 includes hook 264. Side rail 262 includes hook 266. The hooks are coaxial along axis 265 and are dimensioned to engage front rail 214. Axis 265 is generally perpendicular to side rail 260 and side rail 262. During assembly, hooks 264 and 266 are positioned around front rail 214 to connect front support frame 208 to base support frame 206, but allow front support frame 208 to rotate with respect to front rail 214. Side rails 260 and 262 are preferably about 15 inches in length. Top rail 258 is preferably about 24 inches in length. In a preferred embodiment, front support frame 208 is integrally formed.

Axis 235 is generally parallel with axis 255. Axis 265 is generally perpendicular to both axis 235 and axis 255.

Cross brace 276 includes indention 280, serpentine hook 282, indention 278 and serpentine hook 284. Serpentine hook 282 is dimensioned to removably yet securely engage top rail 220 at indention 287. Serpentine hook 284 is dimensioned to removably yet securely engage top rail 240 at indention 288. The hooks, in cooperation, prevent outward movement of support frame 204 with respect to support frame 202. Serpentine hook 282 includes lower bend 283. Serpentine hook 284 includes lower bend 285. Lower bend 283 and lower bend 285, in cooperation, prevent inward movement of support frame 204 with respect to support frame 202.

Clip 268 is connected to side rail 262 adjacent front rail 214 and front stanchion 238 adjacent side rail 216. Clip 270 is connected to side rail 262 adjacent top rail 258 and front stanchion 238 adjacent top rail 240. Clip 274 is secured to side rail 260 adjacent front rail 214 and front stanchion 218 adjacent side rail 212. Clip 272 is secured to side rail 260 adjacent top rail 258 and front stanchion 218 adjacent top rail 220. In another embodiment, clips 268 and 274 are optional.

Referring to FIG. 3B, clip 268 will be further described as an example of the clips preferred. Clip 268 includes side wall 231, center wall 233, and side wall 241. Channel 237 is generally cylindrical and is positioned between side wall 231 and center wall 233. Channel 239 is generally cylindrical and is positioned coaxially with channel 237, between center wall 233 and side wall 241. The channels are dimensioned to releasably secure the side rails with a friction fit. Clip 268 is preferably constructed of a semi-rigid resilient polyethylene Teflon®, Delrin® or polyvinyl chloride.

Referring to FIG. 3C, an exemplary connection between the hooks and the rails of the base support frame will be further described. Hook 254 forms opening 247. Side rail 216 is positioned within opening 247. Opening 247 is dimensioned to retain side rail 216, but allow for rotational movement between front stanchion 238 and side rail 216. Hook 266 forms opening 267. Front rail 214 is positioned in opening 267. Opening 267 is dimensioned to retain front rail 214, but allow for rotation between side rail 262 and front rail 214. Clip 268 is positioned on side rail 262 and front stanchion 238. Side rail 262 fits within channel 237. Front stanchion 238 fits within channel 239. Positioning the clip fixes side rail 262 and front stanchion 238 in an upright and perpendicular position with respect to the base support frame. The connections between front support frame 208, base support frame 206, support frame 204 and support frame 202 are similarly functional and similarly arranged.

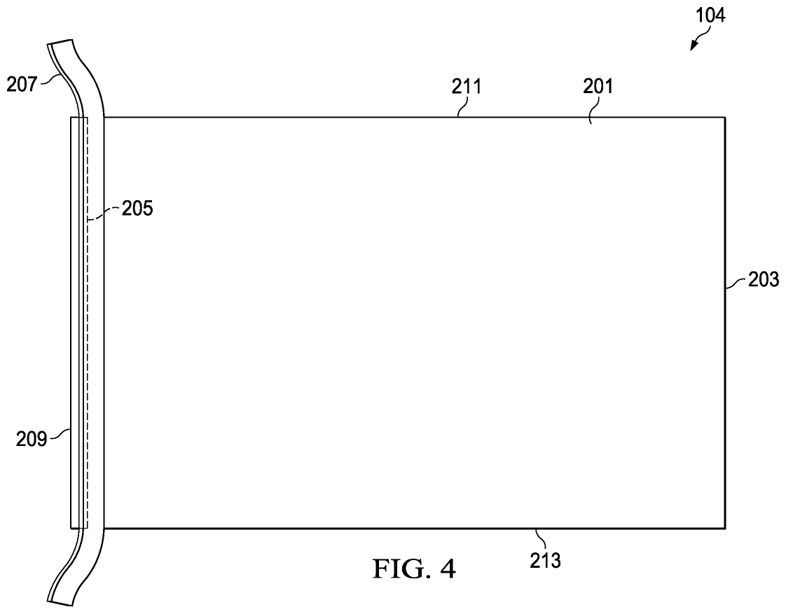

Referring to FIG. 4, drape subassembly 104 will be further described. Drape subassembly 104 includes transparent sheet 201 bonded to flexible seal 205. Transparent sheet 201 is bounded by top edge 203, side edge 213, bottom edge 209, and side edge 211. Flexible seal 205 is positioned adjacent and generally parallel to bottom edge 209. Flexible seal 205 is preferably constructed of closed cell butyl rubber foam, about 2 inches in width and ½ inch in height. Strap 207 is affixed adjacent and generally parallel to flexible seal 205. Transparent sheet 201 is preferably constructed of a clear polyethylene sheeting between about 8 and 10 mils thick. In an alternate preferred embodiment, the transparent sheet is integrally formed with the enclosure subassembly.

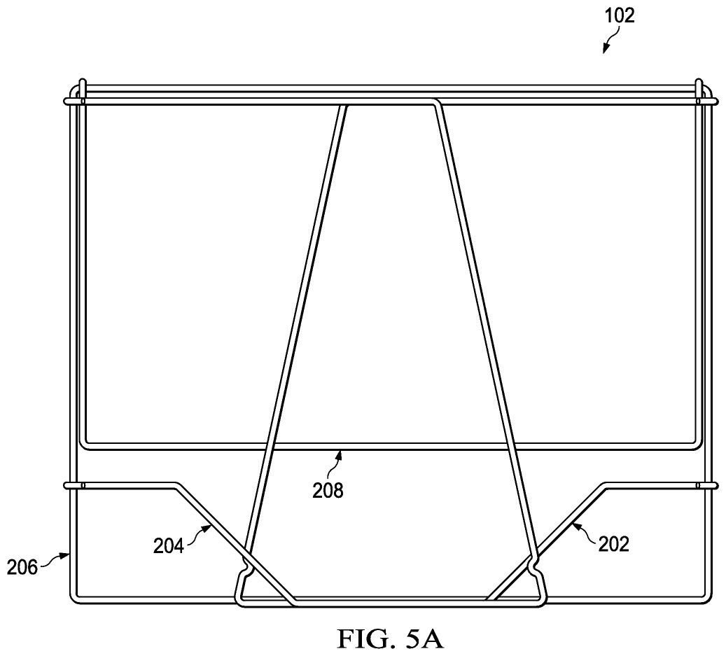

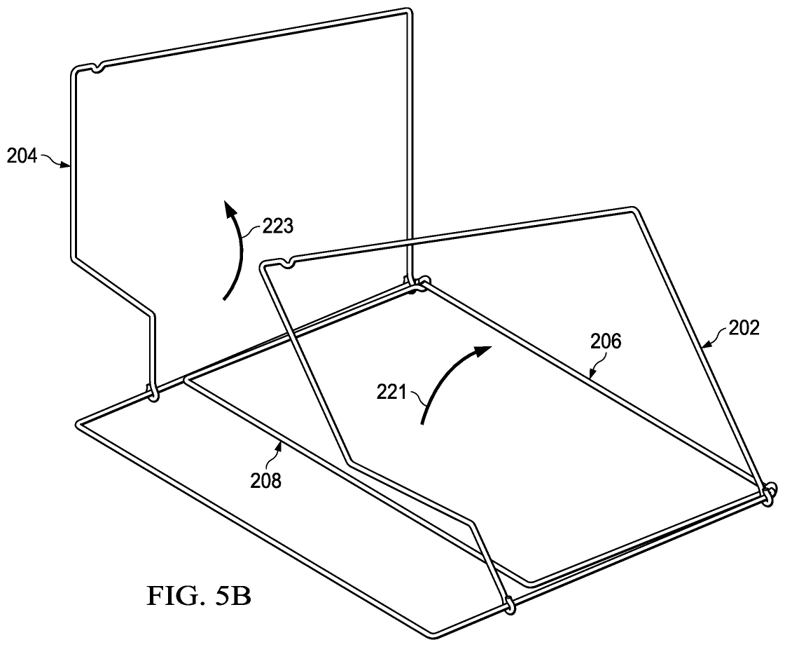

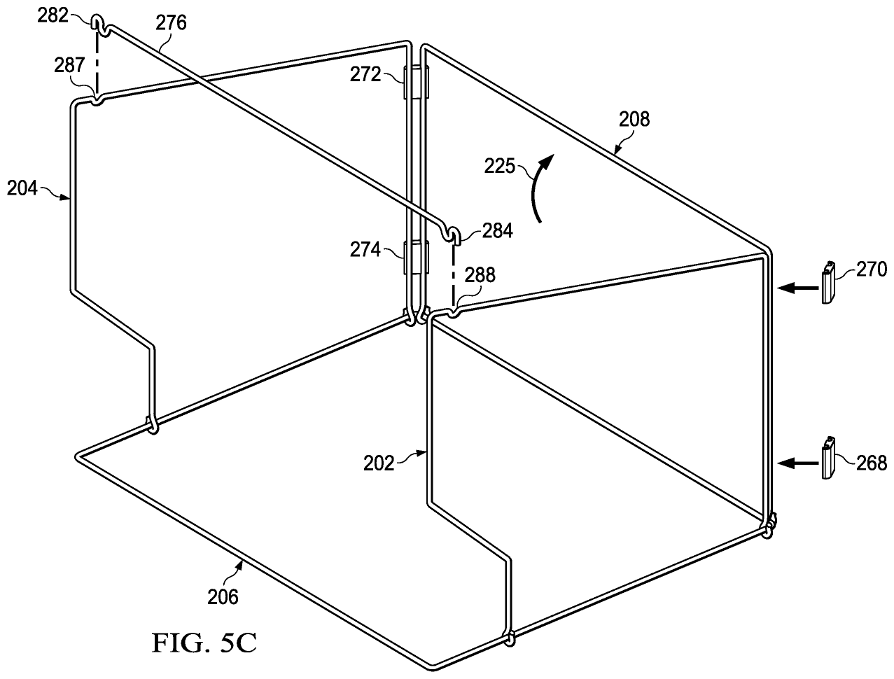

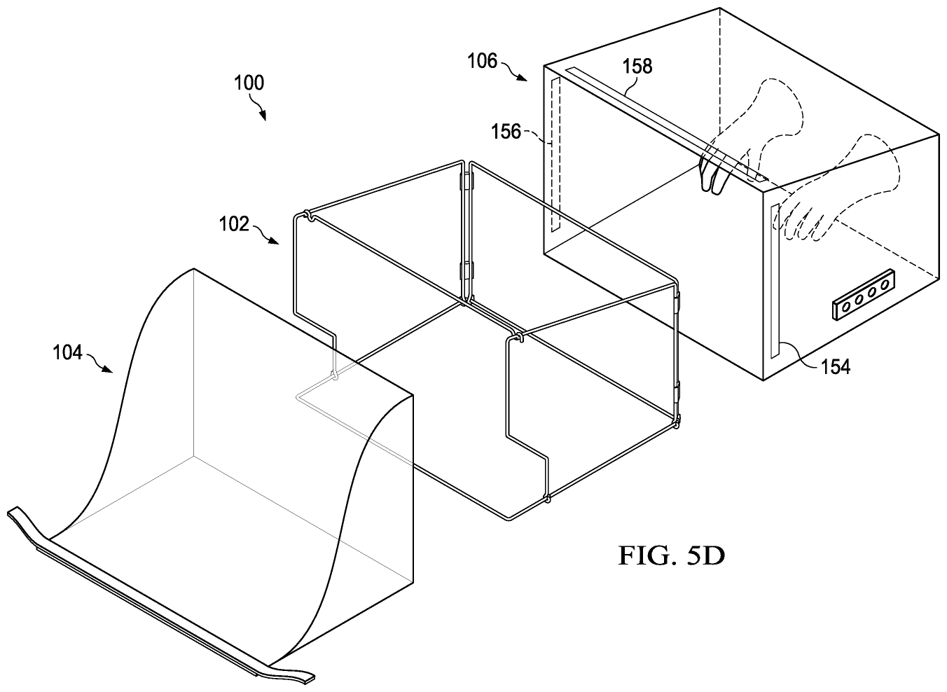

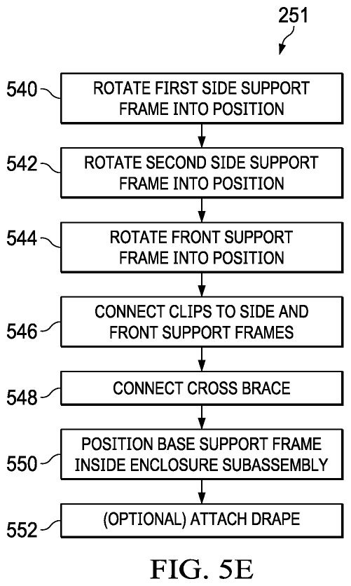

Referring to FIGS. 5A-5E, method 251 of assembly of isolette 100 will be further described. FIG. 5A depicts, frame subassembly 102 in a collapsed configuration. At step 540, support frame 204 is rotated in direction 223 until it is substantially perpendicular to base support frame 206. At step 542, support frame 202 is rotated in direction 221 until it is substantially perpendicular to base support frame 206, and substantially parallel to support frame 204.

At step 544, front support frame 208 is rotated in direction 225 until it is substantially perpendicular to base support frame 206, and substantially perpendicular to both support frame 204 and support frame 202.

At step 546, clip 270 is connected to front support frame 208 and support frame 202. Clip 268 is connected similarly to front support frame 208 and support frame 202. Clip 272 is connected similarly to front support frame 208 and support frame 204. Clip 274 is connected similarly to front support frame 208 and support frame 204.

At step 548, hook 282 is positioned in indent 287 and hook 284 is positioned in indent 288, thereby connecting cross brace 276 on support frames 204 and 202. After this step, the frame subassembly is in a deployed configuration.

At step 550, frame subassembly 102 is placed inside enclosure subassembly 106.

At step 552, drape subassembly 104 is attached to enclosure subassembly 106 by adhesive strips 154, 158, and 156. Optionally, drape subassembly 104 can be integrally formed with the enclosure subassembly 106, obviating the need for this step.

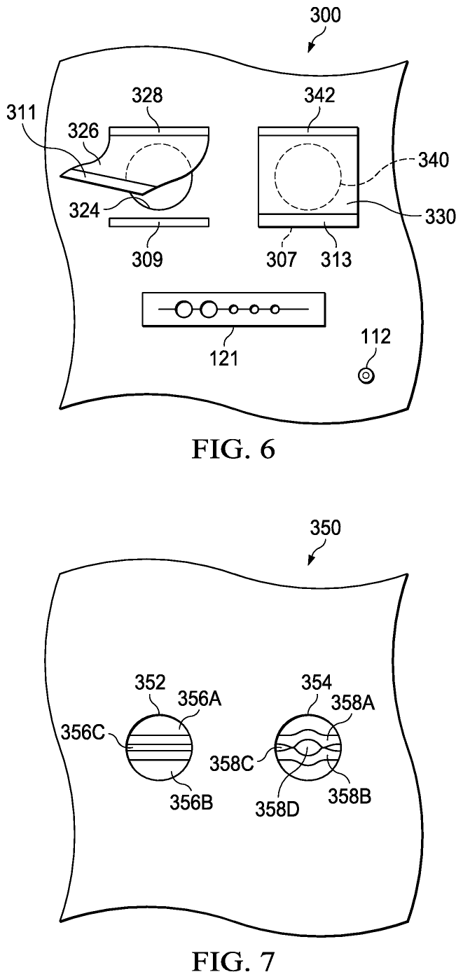

Referring to FIG. 6, an alternate embodiment of a side or front panel of the enclosure, will be further described. All versions of the side or front panels may optionally be employed in all the various embodiments of the isolette. Each of the side and front panels of the various embodiments may include zero, one or two access ports and/or gloves, as will be further described.

Panel 300 further comprises access ports 324 and 340. In this example two (2) access ports, component access panel 121 and pressure fitting 112 are provided in a side panel. However, in other embodiments, a single access port in a side panel may be provided. In yet other embodiments, no access ports are provided in a side panel, but is created as needed on an ad hoc basis, as will be further described. Flap 326 is fixed to panel 300 adjacent to and covering access port 324. Flap 326 is fixed to the panel by adhesive 328. Flap 326 further comprises closure 311. Panel 300 further include closure 309. In use, closure 311 is removably affixed to closure 309, thereby sealing flap 326 over access port 324.

Likewise, flap 330 is positioned adjacent to and covering access port 340. Flap 330 is fixed to panel 300 via adhesive 342. Closure 307 is fixed to panel 300. Closure 313 is fixed to flap 330. In use, closure 313 is adapted to removably seal against closure 307, thereby sealing flap 330 over access port 340.

Referring then to FIG. 7, an alternate embodiment of an enclosure side or front panel will be further described.

Panel 350 is further comprised of access ports 352 and 354. Access port 352 further comprises iris diaphragms 356a and 356b. Iris diaphragm 356a overlaps iris diaphragm 356b, in region 356c.

Access port 354 is further comprised of iris diaphragm 358a and 358b. Iris diaphragm 358a overlaps iris diaphragm 358b, in region 358c. Iris diaphragms 358a and 358b, are shown in an open position, accommodating opening 358d.

In a preferred embodiment, the iris diaphragms are comprised of butyl rubber sheet or low-density polyethylene, approximately 10 mils in thickness. The iris diaphragms are fixed to the exterior of the access port, except for the overlap region, which may be parted by physical pressure, to create a ducted portal in the access port, such as opening 358d.

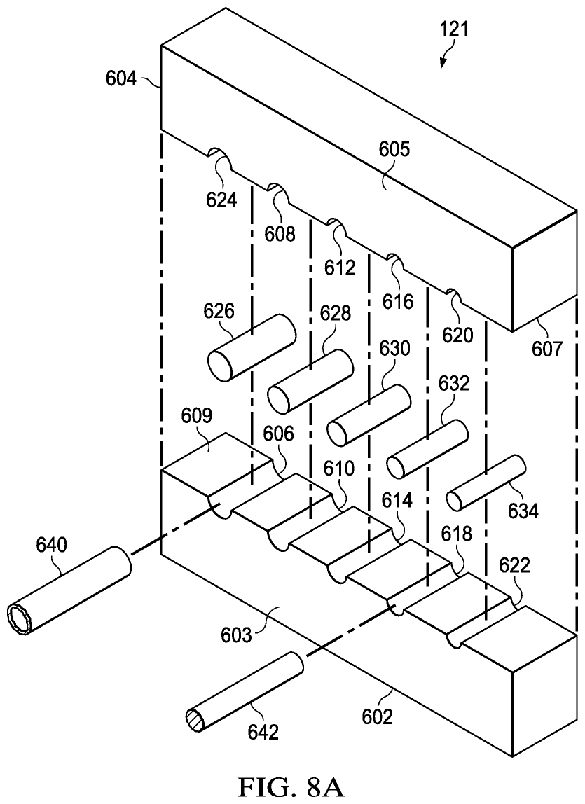

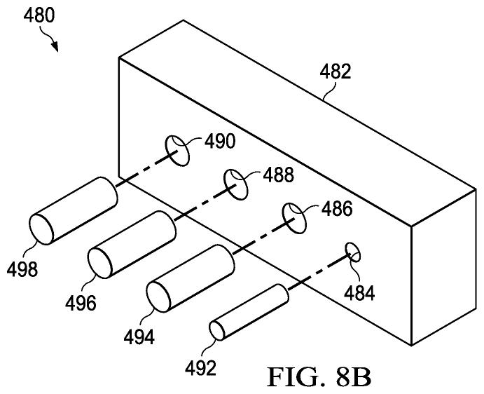

Referring to FIG. 8A, a preferred embodiment of the component access panel will be further described. Component access panel 121 includes top section 604 and bottom section 602. Top section 604 further comprises front face 605 and bottom face 607. Bottom section 602 further comprises front face 603 and top face 609. When assembled, front face 605 and front face 603 are positioned adjacent the inside surface of the side panel and affixed with a suitable medical adhesive. Bottom face 607 is positioned adjacent top face 609.

Semi-cylindrical slots 606, 610, 614, 618, and 622 are formed in the bottom section. Semi-cylindrical slots 624, 608, 612, 616, and 620 are formed in the top section adjacent semi-cylindrical slots 606, 610, 614, 618, and 622, respectively. In general, the slots are all generally parallel and all generally perpendicular to the side panel. When the top and bottom sections are assembled, resealable plug 626 fits within slots 606 and 624. Resealable plug 628 fits within semi-cylindrical slots 608 and 610. Resealable plug 630 fits within semi-cylindrical slots 612 and 614. Resealable plug 632 fits within semi-cylindrical slots 616 and 618. Resealable plug 634 fits within semi-cylindrical slots 620 and 622. The plugs prevent gas leakage through the access panel. In a preferred embodiment, the slots are adapted to seal against the outside surface of different sized tubes and wires, thereby preventing gas leakage through the access panel by insertion of tubes 640 and wires 642, as will be further described. In a preferred embodiment, component access panel 121 is approximately ¼ inch×1 inch cross-section and approximately 8 inches in length and is comprised of closed cell neoprene rubber foam.

Referring to FIG. 8B, an alternate embodiment of the component access panel will be described.

Access panel 480 is comprised of a single integrated block 482. Integrated block 482 includes through holes 484, 486, 488 and 490. Removable plugs 492, 494, 496 and 498 are positioned in through holes 484, 486, 488 and 490. Preferably, the removable plugs are integrally formed with integrated block 482 and held in place by a perforated break line. The block is preferably comprised of closed cell neoprene rubber foam.

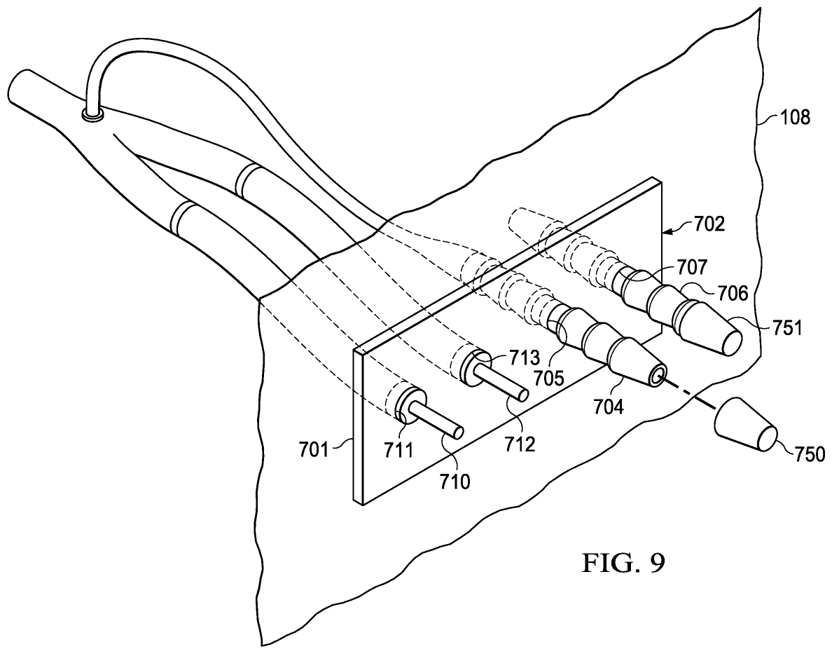

Referring to FIG. 9, an alternate embodiment of the component access panel will be described. Component access panel 702 is generally a semi-rigid plastic connector block supporting several hose connectors. Component access panel 702 further comprises rear surface 701, adhered to side panel 108 with a suitable adhesive.

Component access panel 702 preferably includes a number of double ended connectors forming ducted portals from the exterior of the isolette to its interior. For example, component access panel 702, in this embodiment, includes circuit connector 710, circuit connector 712, circuit connector 704, and circuit connector 706. Circuit connector 710 and circuit connector 712 are adapted to connect to anesthesia lines. Circuit connector 704 and circuit connector 706 are adapted to connect to a source of vacuum. Circuit connector 710 passes through component access panel 702 thru through hole 711. Likewise, circuit connectors 712, 704 and 706 pass through component access panel 702 via through holes 713, 705 and 707, respectively.

In this example, the circuit connectors are adapted to be sealed by caps when not in use, such as cap 750 adapted to mate with circuit connector 704, and cap 751 shown in place on circuit connector 706. In this example, the circuit connectors are either Luer lock type, wedge type, slip type or barb type connectors. Other connector types may be used equally well.

In a preferred embodiment, the component access panel is comprised of a semi flexible polyvinyl chloride sheet, approximately 100 mils thick. The circuit connectors may be adhered in the through holes with a suitable medical grade adhesive sealant.

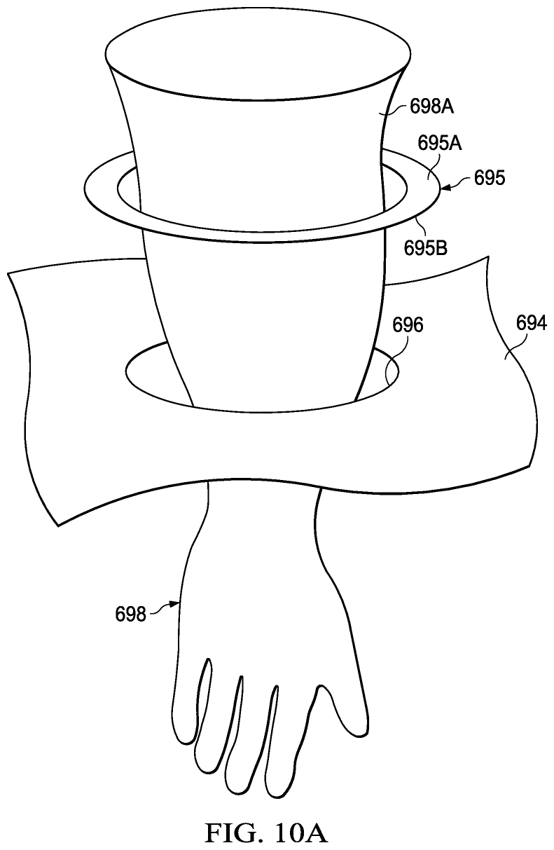

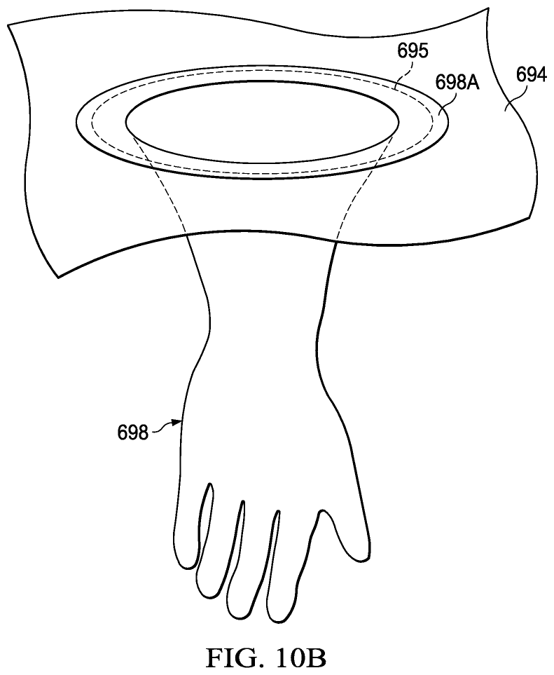

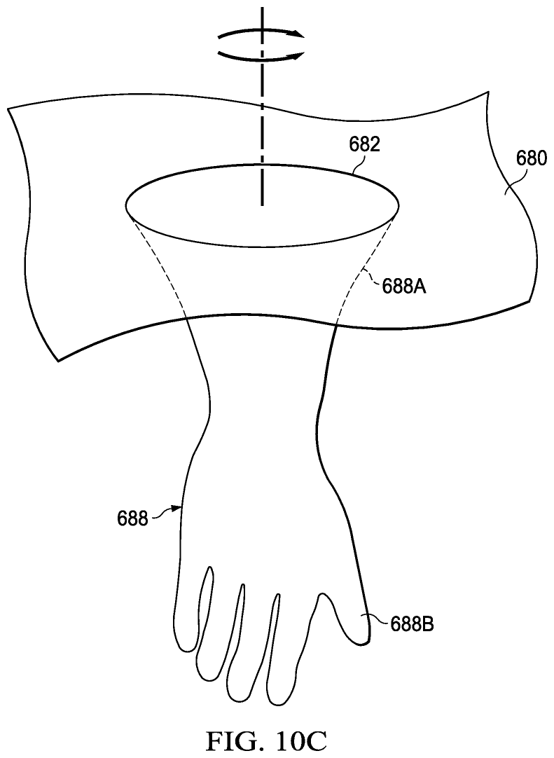

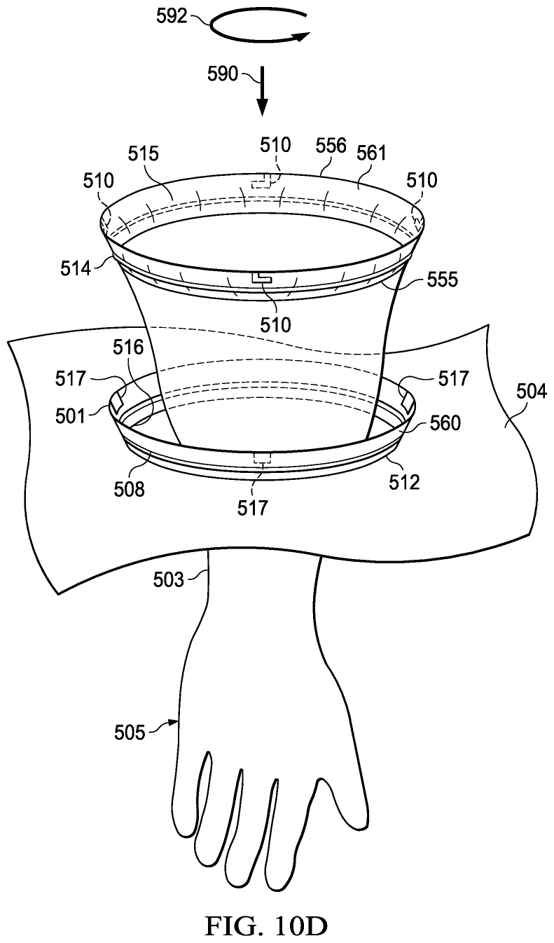

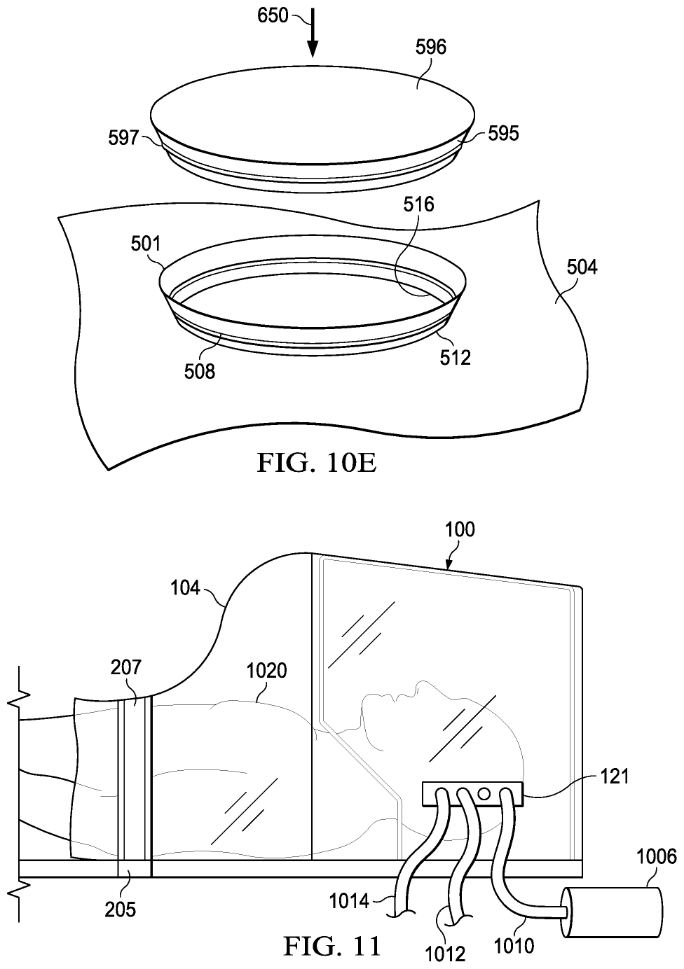

Referring to FIGS. 10A and 10B, an alternate embodiment of an access port will be described.

Panel 694 includes access port 696. Adhesive ring 695 is adapted to circumscribe access port 696. Adhesive ring 695 further comprises adhesive surface 695a and adhesive surface 695b. Glove 698 is adapted to fit within access port 696 and within adhesive ring 695.

Referring then to FIGS. 10A and 10B, when assembled, glove sleeve 698a is fixed to adhesive surface 695a. Panel 694 is fixed to adhesive surface 695b. The connections between glove sleeve 698a, adhesive surface 695a, adhesive surface 695b and panel 694, create an airtight seal, thereby allowing access to the glove from the exterior of the isolette without release of contagions within the isolette.

Referring thing to FIG. 10C, an alternate embodiment of an access port will be described. Panel 680 includes access port 682. Panel 680 is fused to glove sleeve 688a of glove 688, around access port 682, thereby allowing access to the glove from the exterior of the isolette. In this embodiment, glove thumb 688b is oriented vertically up with respect to the isolette, when fused to access port 682. The orientation of the glove is important, because a single “non-handed” glove may be used for either the left, or the right, hand. This embodiment is also important, as will be further described, because it can be placed in any position, in a side, or top panel, on an ad-hoc basis.

Referring to FIG. 10D, an alternate embodiment of an access port will be described. Panel 504 includes connection ring 512 sealed adjacent access port 516 along annular interface 501. The connection ring is sealed at the annular interface with suitable medical adhesive. Connection ring 512 is further comprised of frustroconical body 560. Frustroconical body 560 includes annular O-ring channel 508 on its interior surface. Frustroconical body 560 further includes a plurality of bayonet mount pins 517, dispersed at 90° angles on the interior surface of the frustroconical body relative to a central axis of the frustroconical body.

Glove collar 556 is designed to mate with connection ring 512. Glove collar 556 includes frustroconical body 561. Frustroconical body 561 further includes annular O-ring 514 compressed into exterior annular channel 515. The frustroconical body further includes bayonet mount receptors 510.

Glove 505 is attached to glove collar 556 at sleeve 503 and annular interface 555. In preferred embodiments, sleeve 503 is adhered to annular interface 555 with a suitable medical grade adhesive.

In use, glove 505 is advanced through connection ring 512 and into the interior of the isolette. Glove collar 556 advances in direction 590 into connection ring 512 where annular O-ring 514 seats in O-ring channel 508, thereby forming an air-tight seal. Once the O-ring is seated, glove collar 556 is rotated in direction 592 to lock bayonet mount receptors 510 into position adjacent bayonet mount pins 517.

Referring to FIG. 10E, an alternate embodiment of a connection ring, will be further described. Connection ring 512 includes O-ring channel 508 adjacent its interior annular surface. Barrier cap 596 includes frustroconical body 595 and is generally adapted to seal to the connection ring. Frustroconical body 595 includes annular O-ring 597. O-ring 597 is adapted to seat within O-ring channel 508 as barrier cap 596 is advanced into access port 516 in direction 650. Barrier cap 596 is removably fixed in the access point and creates an airtight seal between the exterior of the isolette and the interior of the isolette.

Referring to FIG. 11, isolette 100 is shown in use. Patient 1020 is positioned inside the isolette on the bottom panel. Drape subassembly 104 is extended from a position above the isolette downward and around the patient. Optional flexible seal 205 is secured around the patient. Optional strap 207 is secured to hold the flexible seal in place. Negative pressure tube 1010 is ductedly connected to component access panel 121 which provides negative pressure to the isolette from filtered negative pressure turbine 1006. Optionally oxygen line 1012 is inserted through component access panel 121 and secured to the patient. Optionally, EKG line 1014 is inserted through component access panel 121 and secured to the patient.

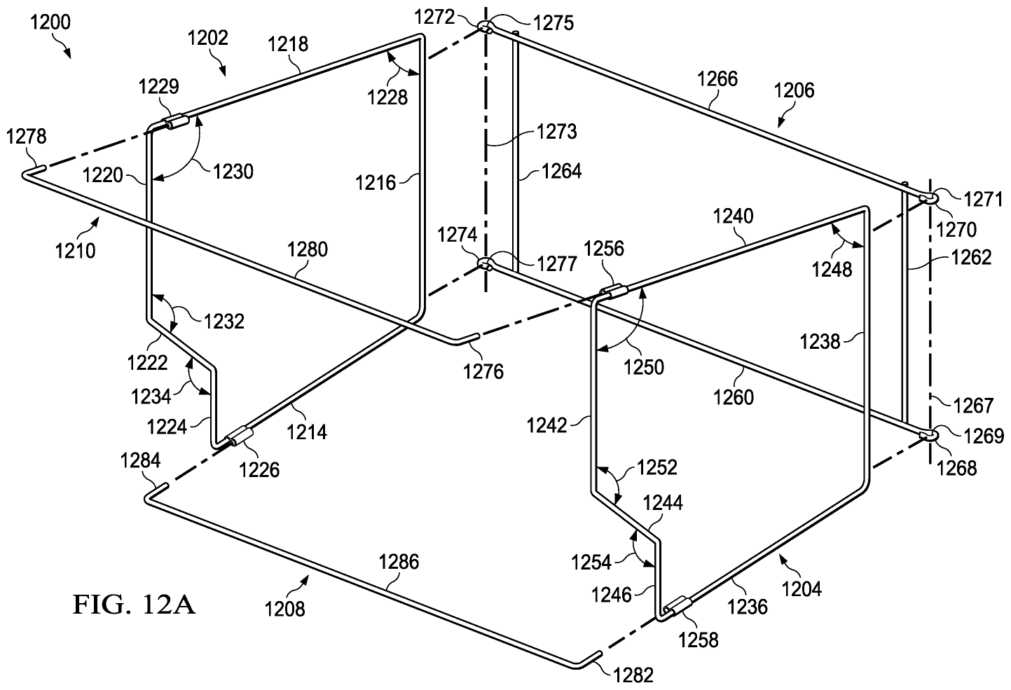

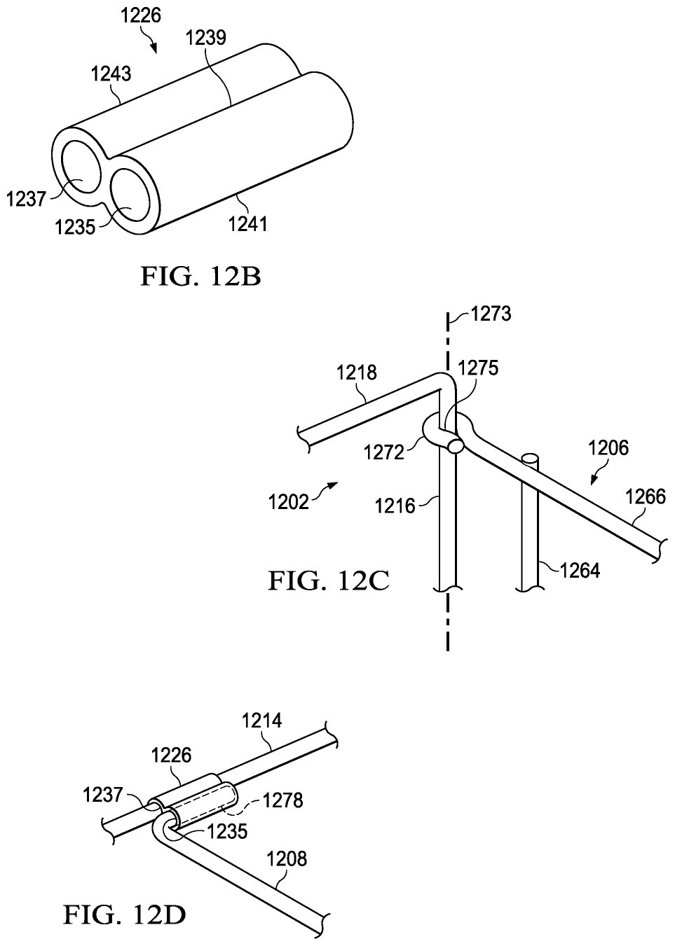

Referring to FIG. 12A, an alternate preferred embodiment of frame subassembly 1200 will be further described. Frame subassembly 1200 includes support frame 1202, support frame 1204, front support frame 1206, cross brace 1208 and cross brace 1210. Preferably frame subassembly 1200 is constructed from approximately 3/16 inch diameter stainless steel or aluminum drawn wire. Alternatively, 3/16 inch to ¼ inch metallic tubing will also serve well. In other embodiments, fiberglass or graphite composite rod stock will also suffice.

Support frame 1202 includes bottom rail 1214, front stanchion 1216, top rail 1218, rear stanchion 1220, angled segment 1222, and vertical segment 1224. Front stanchion 1216 is substantially perpendicular to bottom rail 1214. Front stanchion 1216 is connected to top rail 1218 at angle 1228 which is preferably about 97°. Top rail 1218 is connected to rear stanchion 1220 at angle 1230 which is preferably about 83°. Rear stanchion 1220 is connected to angled segment 1222 at angle 1232 which is preferably about 135°. Angled segment 1222 is connected to vertical segment 1224 at angle 1234 which is preferably about 135°. Vertical segment 1224 is substantially perpendicular to bottom rail 1214. Top rail 1218 includes connector 1229 adjacent rear stanchion 1220. Bottom rail 1214 includes connector 1226 adjacent vertical segment 1224. In one embodiment, the connectors are tack welded in place. In another embodiment, the connectors are free to slide longitudinally along the rails to permit positional adjustment of the attached cross brace. Preferably, bottom rail 1214, front stanchion 1216, top rail 1218, rear stanchion 1220, angled segment 1222, and vertical segment 1224 are integrally formed.

Support frame 1204 includes bottom rail 1236, front stanchion 1238, top rail 1240, rear stanchion 1242, angled segment 1244, and vertical segment 1246. Front stanchion 1238 is substantially perpendicular to bottom rail 1236. Front stanchion 1238 is connected to top rail 1240 at angle 1248 which is preferably about 97°. Top rail 1240 is connected to rear stanchion 1242 at angle 1250 which is preferably about 83°. Rear stanchion 1242 is connected to angled segment 1244 at angle 1252 which is preferably about 135°. Angled segment 1244 is connected to vertical segment 1246 at angle 1254 which is preferably about 135°. Vertical segment 1246 is substantially perpendicular to bottom rail 1236. Top rail 1240 includes connector 1256 adjacent rear stanchion 1242. Bottom rail 1236 includes connector 1258 adjacent vertical segment 1246. In one embodiment, the connectors are tack welded in place. In another embodiment, the connectors are free to slide longitudinally along the rails to permit positional adjustment of the attached cross brace. Preferably, bottom rail 1236, front stanchion 1238, top rail 1240, rear stanchion 1242, angled segment 1244, and vertical segment 1246 are integrally formed.

In a preferred embodiment, front stanchions 1216 and 1238 are about 15 inches in length, top rails 1218 and 1240 are about 15 inches in length, rear stanchions 1220 and 1242 are about 9 inches in length, angled segments 1222 and 1244 are about 6 inches in length, and vertical segments 1224 and 1246 are about 4 inches in length. Bottom rails 1214 and 1236 are about 10 inches in length.

Front support frame 1206 includes top rail 1266, side rail 1262, side rail 1264, and bottom rail 1260. Side rail 1264 is substantially perpendicular to top rail 1266 and bottom rail 1260. Side rail 1262 is substantially perpendicular to top rail 1266 and bottom rail 1260. Top rail 1266 includes hook 1272 and hook 1270. Hooks 1272 and 1270 include generally circular openings 1275 and 1271, respectively. Opening 1275 is positioned around front stanchion 1216, adjacent top rail 1218. Opening 1271 is positioned around front stanchion 1238, adjacent top rail 1240. Bottom rail 1260 includes hook 1274 and hook 1268. Hooks 1274 and 1268 include generally circular openings 1277 and 1269, respectively. Opening 1277 is positioned around front stanchion 1216, adjacent bottom rail 1214. Opening 1269 is positioned around front stanchion 1238, adjacent bottom rail 1236. Hooks 1272 and 1274 are coaxial about axis 1273, and connect front support frame 1206 to support frame 1202, while allowing front stanchion 1216 to rotate about that axis. Hooks 1270 and 1268 are coaxial about axis 1267, and connect front support frame 1206 to support frame 1204 while allowing front stanchion 1238 to rotate about that axis. Axis 1267 and axis 1273 are generally parallel. Top rail 1266 and bottom rail 1260 are about 24 inches in length. Side rails 1264 and 1262 are about 14 inches in length.

Cross brace 1210 includes rail 1280, and anchor pins 1276 and 1278. Anchor pins 1276 and 1278 are substantially perpendicular to rail 1280. Cross brace 1208 includes rail 1286 and anchor pins 1282 and 1284. Anchor pins 1282 and 1284 are substantially perpendicular to rail 1286. Cross brace 1208 is optional. Rails 1280 and 1286 are about 24 inches in length. Anchor pins 1276, 1278, 1282, and 1284 are about 1½ inches in length. In one preferred embodiment, the anchor pins may be knurled to prevent easy extraction from the connectors. The cross braces are generally parallel to front support frame 1206 and generally perpendicular to both support frames 1202 and 1204.

Referring to FIG. 12B, connector 1226 will be described as an example of all the connectors. Connector 1226 includes parallel tubes 1243 and 1241. Tubes 1243 and 1241 are preferably tack welded along connection 1239. Alternatively, the connectors may be integrally formed or cast. Tube 1243 includes hole 1237. Tube 1241 includes hole 1235. Hole 1237 and hole 1235 are generally parallel. Preferably, holes 1237 and 1235 are of a diameter slightly larger than the diameter bottom rail 1214, which they may accommodate with a loose friction fit. Connectors 1229, 1256, and 1258 are of similar construction to connector 1226.

Referring to FIG. 12C, the rotational connection between front support frame 1206 and support frame 1202 will be further described as an example of all the rotational connections. Hook 1272 forms opening 1275 which is positioned around front stanchion 1216. Opening 1275 is generally circular and is of a sufficient diameter to allow free rotation of front stanchion 1216 about axis 1273, but yet retain the front stanchion in the opening. The rotational connections of hooks 1274, 1270, and 1268 with respect to front stanchions 1216 and 1238, are similar and function in a similar way.

Referring then to FIG. 12D, the connection between connector 1226, bottom rail 1214 and cross brace 1208 will be described as an example of the other connections. Hole 1237 is fitted on bottom rail 1214 with a preferably loose friction fit which allows the connector to slide along the bottom rail. In alternate embodiments, connector 1226 may be spot welded to bottom rail 1214. Hole 1235 is dimensioned to create a friction fit with anchor pin 1278 sufficient to prevent easy extraction of the cross brace from the connector.

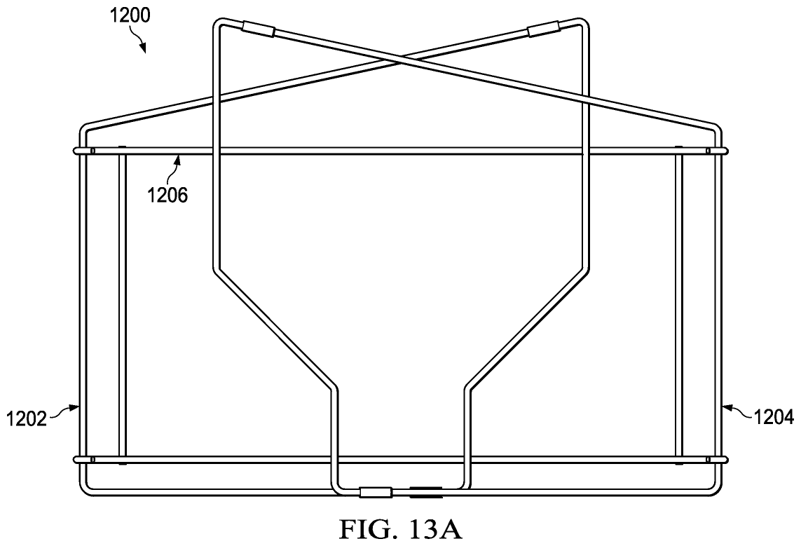

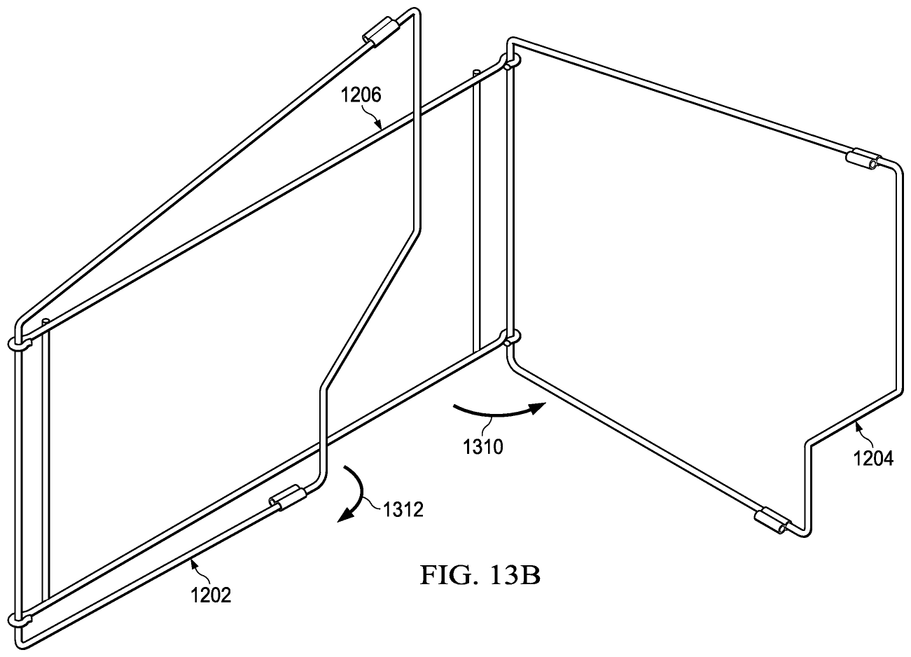

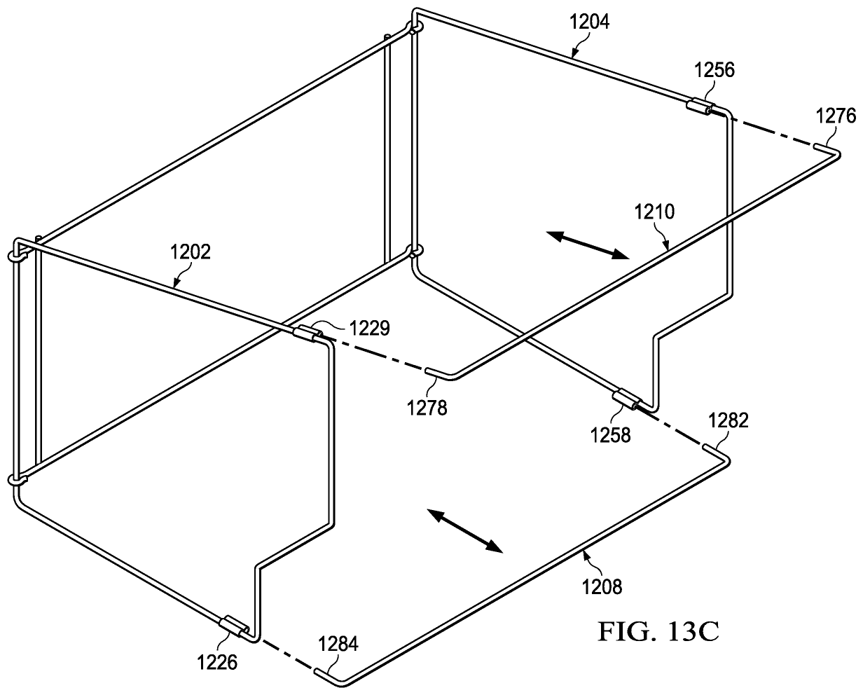

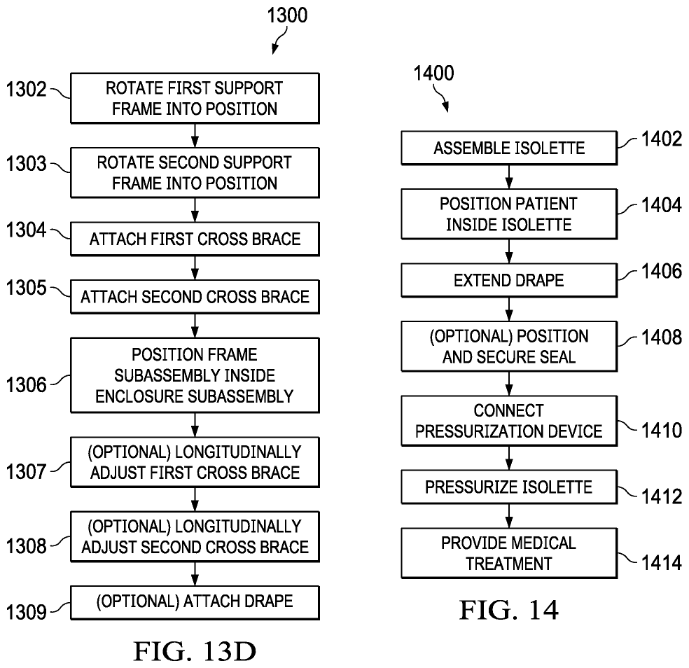

Referring to FIGS. 13A-13D, method 1300 of assembly of the isolette is further described. FIG. 13A depicts frame subassembly 1200 in a collapsed configuration. At step 1302, support frame 1204 is rotated in direction 1310 until support frame 1204 is substantially perpendicular to front support frame 1206. At step 1303, support frame 1202 is rotated in direction 1312 until support frame 1202 is substantial perpendicular to front support frame 1206.

At step 1304, anchor pin 1278 is inserted into connector 1229 and anchor pin 1276 is inserted into connector 1256. At step 1305, anchor pin 1284 is inserted into connector 1226 and anchor pin 1282 is inserted into connector 1258. Insertion of the anchor pins into the connections fixes support frames 1202 and 1204 generally parallel to each other and generally perpendicular to front support frame 1206. After this step, the frame subassembly is in a deployed configuration.

At step 1306, frame subassembly 1200 is inserted into enclosure subassembly 106, as previously described.

At step 1307, optionally, the upper cross brace is adjusted longitudinally with respect to the support frames. At step, 1308, optionally, the lower cross brace is adjusted longitudinally with respect to the support frames. The adjustments are important because they provide for patient comfort and customization of the frame assembly.

At step 1309, drape subassembly 104 is attached to enclosure subassembly 106 as previously described. The drape assembly may be integrally formed with the enclosure, making this step unnecessary.

Referring to FIG. 14, a preferred method of use 1400 of an isolette as disclosed will be described.

At step 1402, the isolette is assembled as previously described. The assembled isolette is positioned on a flat surface, such as a bed, gurney, or operating table.

At step 1404, the patient is positioned inside the isolette in a supine position. Preferably, the patient's head is located adjacent the front panel and below the top panel.

At step 1406, the drape or transparent sheet is extended over the upper torso of the patient.

At step 1408, the optional flexible seal is fitted around the patient's arms and torso. The optional flexible seal is then secured in position. In a preferred embodiment, the seal is held in place with a strap attached to the table, bed, or gurney. In another preferred embodiment, the seal is held in position with medical adhesive tape.

At step 1410, the filtered negative pressure turbine is connected to the isolette through the connector.

At step 1412, pressurization is applied to the isolette. In a preferred embodiment, the isolette is capable of supporting negative pressure greater than 0.01 mmHg. In the unusual situation where a positive internal pressure is required, the isolette will support it. In this case, at this step, the positive pressure is applied.

At step 1414, medical treatment is provided to the patient through use of the access ports and optional component access panel. The preferred methods of providing medical treatment have been previously discussed.

It will be appreciated by those skilled in the art that changes could be made to the embodiments described above without departing from the broad inventive concept thereof. It is understood, therefore, that this disclosure is not limited to the particular embodiments disclosed, but it is intended to cover modifications within the spirit and scope of the present disclosure as defined by the appended claims.

1. A medical isolette comprising:
a base frame;
a first support frame;
a first pair of openings, formed in the first support frame, and rotatably attached to the base frame;
a second support frame;
a second pair of openings, formed in the second support frame, and rotatably attached to the base frame;
a third support frame;
a third paid of openings, formed in the third support frame, and rotatably attached to the base frame;
a removable cross brace, connecting the first support frame and the second support frame;
a flexible membrane, having a drape section, removably enclosing the base frame, the first support frame, the second support frame, and the third support frame; and,
the drape section forming a generally sealable isolation chamber.

2. The medical isolette of claim 1 further comprising:
a set of access ports, fixed in the flexible membrane;
a set of medical gloves, sealed into the set of access ports, projectable into the sealable isolation chamber.

3. The medical isolette of claim 2 wherein the set of medical gloves is removable.

4. The medical isolette of claim 1 further comprising:
a semi-rigid access panel, sealed into the flexible membrane; and,
a set of sealable portals, in the semi-rigid access panel, ductedly connected to the sealable isolation chamber.

5. The medical isolette of claim 1 wherein:
a first retainer clip, releasably joining the first support frame and the third support frame; and,
a second retainer clip, releasably joining the second support frame and the third support frame.

6. The medical isolette of claim 1 wherein:
each opening, of the first pair of openings, is coaxial along a first axis;
each opening, of the second pair of openings, is coaxial along a second axis;
each opening, of the third pair of openings, is coaxial along a third axis;
the first axis and the second axis are generally parallel; and,
the third axis is generally perpendicular to the first axis and the second axis.

7. The medical isolette of claim 1 further comprising:
a source of negative gas pressure, ductedly connected to the sealable isolation chamber, through the flexible membrane.

8. The medical isolette of claim 1 wherein the drape section further comprises:
a transverse flexible sealing strip adjacent a securing strap.

9. The medical isolette of claim 1 wherein the flexible membrane slopes toward the third support frame.

10. The medical isolette of claim 1 wherein:
the first pair of openings forms a first set of hooks coaxially surrounding a first rail of the base frame;
the second pair of openings forms a second pair of hooks coaxially surrounding a second rail of the base frame; and,
the third pair of openings forms a third pair of hooks coaxially surrounding a third rail of the base frame.

11. The medical isolette of claim 1 wherein the base frame, the first support frame, the second support frame and the third support frame are of wire frame construction.

12. A method of constructing a medial isolette comprising:
rotating a first side support frame upward from a base frame;
rotating a second side support frame upward from the base frame;
rotating a front support frame upward from the base frame;
securing the first side support frame to the front support frame with a first resilient connector;
securing the second side support frame to the front support frame with a second resilient connector;
removably securing a cross brace between the first side support frame and the second side support frame; and,
positioning the base frame, the first side support frame, the second side support frame and the front support frame in a form fitting flexible enclosure to form a generally sealable isolation chamber.

13. The method of claim 12 further comprising:
attaching a drape subassembly to the enclosure adjacent the cross brace.

14. The method of claim 12 further comprising:
providing an access port in the flexible enclosure, ductedly connected to the sealable isolation chamber.

15. The method of claim 14 further comprising:
sealing a medical glove in the access port projecting into the sealable isolation chamber.

16. The method of claim 12 further comprising:
ductedly connecting a negative gas pressure source to the sealable isolation chamber through the flexible enclosure.

17. The method of claim 12 further comprising:
positioning a ported access panel in the flexible enclosure; and,
opening at least one port in the ported access panel to create a ducted connection to the sealable isolation chamber.

18. A medical isolette comprising:
a front support frame;
a first pair of coaxial openings connected to the front support frame;
a second pair of coaxial openings connected to the front support frame;
a first side support frame, rotatably positioned in the first pair of coaxial openings;
a second side support frame, rotatably positioned in the second pair of coaxial openings;
a top cross brace, removably attached between the first side support frame and the second side support frame;
a bottom cross brace, generally parallel with the top cross brace, removably attached between the first side support frame and the second side support frame;
a flexible enclosure, having a drape section, surrounding the front support frame, the first side support frame, the second side support frame, the top cross brace and the bottom cross brace; and,
the drape section forming a generally sealed isolation chamber.

19. The medical isolette of claim 18 further comprising:
a set of access ports, fixed in the flexible enclosure;
a set of medical gloves, sealed into the set of access ports, projectable into the sealed isolation chamber.

20. The medical isolette of claim 19 wherein the set of medical gloves is removable.

21. The medical isolette of claim 18 further comprising:
a semi-rigid access panel, sealed into the flexible enclosure; and,
a set of sealable portals, in the semi-rigid access panel, ductedly connected to the isolation chamber.

22. The medical isolette of claim 18 wherein:
the front support frame is generally perpendicular to the first side support frame and the second side support frame; and,
the first side support frame is generally parallel to the second side support frame.

23. The medical isolette of claim 18 wherein:
the first side support frame further comprises a first upper connector and a first lower connector;
the second side support frame further comprises a second upper connector and a second lower connector;
the top cross brace is fitted in the first upper connector and the second upper connector; and,
the bottom cross brace is fitted in the first lower connector and the second lower connector.

24. The medical isolette of claim 23 wherein:
the first upper connector and the first lower connector are longitudinally movable with respect to the first side support frame; and,
the second upper connector and the second lower connector are longitudinally movable with respect to the second side support frame.

25. The medical isolette of claim 18 wherein:
the first pair of coaxial openings forms a first set of hooks coaxially surrounding a vertical rail of the first support frame; and,
the second pair of coaxial openings forms a second set of hooks coaxially surrounding a vertical rail of the second support frame.

26. The medical isolette of claim 18 further comprising:
a source of negative gas pressure, ductedly connected to the sealed isolation chamber, through the flexible enclosure.

27. The medical isolette of claim 18 wherein the drape section further comprises:
a transverse flexible sealing strip adjacent a securing strap.

28. The medical isolette of claim 18 wherein the flexible membrane slopes toward the front support frame.

29. The medical isolette of claim 18 wherein the front support frame, the first side support frame and the second side support frame are of wire frame construction.

30. A method of constructing a medical isolette comprising:
rotating a first side support frame away from a front support frame;
rotating a second side support frame away from the front support frame;
removably securing a first cross brace between the first support frame and the second support frame; and,
positioning the first side support frame, the second side support frame, the front support frame and the first cross brace in a form fitting flexible enclosure to form a generally sealable isolation chamber.

31. The method of claim 30 further comprising:
removably securing a second cross brace between the first support frame and the second support frame.

32. The method of claim 31 further comprising:
longitudinally adjusting the first cross brace; and,
longitudinally adjusting the second cross brace.

33. The method of claim 30 further comprising:
attaching a drape subassembly to the flexible enclosure adjacent the first cross brace.

34. The method of claim 30 further comprising:
providing an access port in the flexible enclosure, ductedly connected to the sealable isolation chamber.

35. The method of claim 34 further comprising:
sealing a medical glove in the access port, projecting into the sealable isolation chamber.

36. The method of claim 30 further comprising:
ductedly connecting a negative gas pressure source to the sealable isolation chamber through the flexible enclosure.

37. The method of claim 30 further comprising:
positioning a ported access panel in the flexible enclosure; and,
opening at least one port in the ported access panel to create a ducted connection to the sealable isolation chamber.
